(19) United States
(12) Patent Application Publication
(10) Pub. No.: US 2023/0183675 A1
(43) Pub. Date: Jun. 15, 2023

(54) Synthetic Single Domain Library

(71) Applicants: Institut Curie, Paris (FR); Centre National de la Recherche Scientifique, Paris (FR)

(72) Inventors: Franck Perez, Paris (FR); Sandrine Moutel, Paris (FR)

(73) Assignees: Institut Curie, Paris (FR); Centre National de la Recherche Scientifique, Paris (FR)

(21) Appl. No.: 17/925,401

(22) PCT Filed: May 20, 2021
(86) PCT No.: PCT/EP2021/063502

§ 371 Date: Nov. 15, 2022

(30) Foreign Application Priority Data
May 20, 2020 (EP) 20305536.3

(51) Int. Cl. C12N 15/10; C07K 16/00; C07K 16/28

(52) U.S. Cl. CPC C12N 15/1037; C07K 16/005; C07K 16/2863; C07K 2317/569; C07K 2317/565; C07K 2317/567; C07K 2317/92; C07K 2317/24

(57) ABSTRACT

The present invention relates to the identification of a fully humanized single domain antibody scaffold as well as its use in generating synthetic single domain antibodies. The invention further relates to antigen-binding proteins comprising said single domain antibody scaffold and their use in therapy.

### FIELD OF THE INVENTION

The invention relates to the identification of a highly stable synthetic single domain antibody scaffold and its use in generating synthetic single domain antibody libraries. The invention also relates to antigen-binding proteins comprising said stable single domain antibody scaffold and their uses, in particular as therapeutics notably for the treatment of cancer.

### BACKGROUND OF THE INVENTION

Over the past decade antibodies imposed themselves as one of the most promising therapeutic approaches, in particular in the field of oncology, as well as an important source of research or diagnosis tools.

The Immunoglobulin G (IgG) is the basic structure of a typical antibody, comprising two heterodimers of heavy and light chains bond together by disulphide bridge. Natural single chain antibodies have however been discovered in at least two groups of animals: Camelidae (Hamers-Casterman et al, 1993, Nature, 363, pp 446-448) and sharks (Greenberg et al, Nature. 1995, Mar. 9; 374(6518): 168-73). These single chain antibodies constitute an additional class of IgG devoid of light chain. The recognition part of these single chain natural antibodies includes only the variable domain of the heavy chain called VHH. VHHs contain four frameworks (FR) that form the scaffold of the IgG domain and three complementarity-determining regions (CDRs) that are involved in antigen binding.

Many advantages of VHHs scaffold have been reported: without interchain disulfide bridges, they are generally more soluble and stable in a reducing environment (Wesolowski et al, 2009 Med Microbiol Immunol. August; 198(3): 157-74). VHH have also been reported to have higher solubility, expression yield and thermostability due to their small size (15 kDa) (Jobling S A et al, Nat Biotechnol. 2003 January; 21 (1): 77-80). Moreover, VHH frameworks show a high sequence and structural homology with human VH domains of familly III (Muyldermans et al, 2001. J Biotechnol. June; 74 (4): 277-302) and VHHs have comparable immunogenicity as human VH and thus constitute very interesting agents for therapeutic applications.

The properties of VHH scaffolds have many advantages, for use in therapy: they have a better penetration in tissues, a faster clearance in kidneys, a high specificity but also reduced immunogenicity.

Camelid antibody libraries have been described for example in US2006/0246058 (National Research Council of Canada). The described phage display library comprises fragments of llama antibodies, and especially single domain fragments of variable heavy chains (VHH and VH). The libraries were made using lymphocyte genomes of non-immunized animals (naive library). The resulting phage display library also contains contaminants of conventional VH antibody fragments.

U.S. Pat. No. 7,371,849 (Institute For Antibodies Co., Ltd) also reports methods of making VHH library from VHH genes of camelids. The diversity of such library was obtained by improving the conventional process of isolating VHH variable regions from naive repertoire. However, these prior arts do not address the issue of immunogenicity from non-human derived antibodies. Even if some of them are identified to bind specific target of interest, they cannot be administered in patients for use as therapeutics without the risk of activating the human immune system.

A method to humanize a camelid single-domain antibody is described in Vincke et al, 2008, JBC Vol 284(5) pp 3273-3284.

U.S. Pat. No. 8,367,586 discloses a collection of synthetic antibodies or their fragments. These antibodies comprise variable heavy chain and variable light chain pairs and have, in their framework region, a part of optimal germline gene sequences. This incorporation of human sequence allows to decrease the risk of immunogenicity for therapeutic use.

Monegal et al (2012, Dev Comp Immunol 36(1): 150-6) reports that single domain antibodies with VH hallmarks are repeatedly identified during biopanning of llama naive libraries. In fact, VH hallmarks are more frequently identified on the binders selected from VHH naive library, than VHH hallmarks. For example, Monegal et al have shown that 5% of VH hallmarks are found in the naive library, while 20% of these VH hallmarks are found among the antibodies selected following biopanning against antigens.

Recently, Moutel et al (eLife 2016; 5:e16228) and WO2015063331 disclosed a synthetic library of humanized nanobodies providing functional high affinity antibodies and intrabodies.

However, despite this knowledge, there is still a need to provide further single domain antibody libraries with improved humanization, while preserving single domain specificities and advantages, in particular their high solubility and expression yield.

Accordingly, one aspect of the invention is to provide a fully humanized, recombinant single domain antibody libraries, of high diversity, capable of generating highly stable single domain antibodies with high affinity against specific antigen. Another aspect is to provide a library enriched in single domain antibodies active in the intracellular environment. Yet another aspect is to provide a library enriched in single domain antibodies with high thermostability. Typically, the single domain antibodies obtained as per the present disclosure have also high expression yield. Typically also, said single domain antibodies can overcome the classical technical issues of mAbs such as slow blood clearance, restricted penetration of solid tumors, non-specific uptake by health tissue and inability to access recessed epitopes

### SUMMARY OF THE INVENTION

On ten amino acids differing from human, four hallmark aminoacids of VHH have been identified in the framework-2 region of VHH. The inventors have surprisingly discovered that these 4 camelid hallmarks can replaced by 4 typical human hallmarks, while preserving VHH properties. The result provided herein show that the herein disclosed synthetic single domain antibody library allows obtaining synthetic humanized sdAb having affinity for their target in the nano/pico-molar range which are highly specific. sdAb directed against various cellular targets have been obtained that can be used as intrabodies for intracellular labeling of living cells. Said sdAb can be used to stain target cells. Further, they are able to inhibit downstream activation of their target (i.e., FGFR4 pathway). Said sdAb can also be used to deliver payload to target cells, arm T cell and destroy targeted cells using CAR-T cell approaches. These results therefore show evidence that the present synthetic single domain antibody library provides single domain antibodies highly relevant for cell labeling, diagnostic and therapeutic applications.

The purposes of the invention are achieved by a method of making a synthetic single domain antibody library, said method comprising the steps of:
i) introducing a diversity of nucleic acids encoding CDR1, CDR2, and CDR3, between the respective framework coding regions of a humanized synthetic single domain antibody (which may be referred to as “hs2dAb” hereafter) to generate a diversity of nucleic acids encoding synthetic single domain antibodies with the same synthetic single domain scaffold amino acid sequence;
   wherein said synthetic single domain antibody scaffold amino acid sequence contains at least the following amino acid residues: FRW2-V4, FRW2-G11, FRW2-L12, FRW2-W14.

In some embodiments, the single domain antibody scaffold is derived from Llama species.

In some embodiments, the synthetic single domain antibody (hs2dAb) as herein disclosed further comprises at least one amino acid residue selected from the group consisting of
FRW1-V5, FRW1-E6, FRW1-L11, FRW3-V35, FRW4-R2, and FRW4-L7; and/or
FRW1-P14, FRW3-S17, FRW3-R29, FRW3-A30, FRW2-S16, FRW3-K18, FRW3-V21, FRW3-Y22, FRW3-L23, and FRW3-S27, notably the following combination FRW1-P14, FRW2-S16, FRW3-S17, FRW3-R29, and FRW3-A30.

In some embodiments, the synthetic single domain scaffold amino acid sequence contains at least one of the following amino acid residues FRW2-V4, FRW2-G11, FRW2-L12, FRW2-W14, FRW1-V5, FRW1-E6, FRW1-L11, FRW3-V35, FRW4-R2, FRW4-L7 and optionally further comprising one or more of the following residues FRW1-P14, FRW3-S17, FRW3-R29, FRW3-A30, FRW2-S16, FRW3-K18, FRW3-V21, FRW3-Y22, FRW3-L23, FRW3-S27.

In some embodiments, the synthetic single domain scaffold amino acid sequence contains the following amino acid residues FRW2-V4, FRW2-G11, FRW2-L12, FRW2-W14, FRW1-V5, FRW1-E6, FRW1-L11, FRW3-V35, FRW4-R2, FRW4-L7, FRW1-P14, FRW3-S17, FRW3-R29, FRW3-A30, FRW2-S16, FRW3-K18, FRW3-V21, FRW3-Y22, FRW3-L23, FRW3-S27.

In some of the above-mentioned embodiments, the synthetic single domain antibody further comprises at least one of the amino acid residues selected from the group consisting of FRW2-V5, FRW3-V21 and FRW4-R2.

In some embodiments, the synthetic single domain antibody comprises the following framework regions consisting of FRW1 of SEQ ID NO: 1, FRW2 of SEQ ID NO:2, FRW3 of SEQ ID NO: 3 and FRW4 of SEQ ID NO:4, or functional variant framework regions, for example with no more than 1, 2 or 3 conservative amino acid substitutions within each framework region. In some of these embodiments, the synthetic single domain antibody scaffold contains at least the amino acid residues consisting of FRW2-V4, FRW2-G11, FRW2-L12, and FRW2-W14. In even more specific embodiments, the scaffold comprises at least one of the amino acid residues from the group consisting of FRW2-V5, FRW3-V21 and FRW4-R2.

In one preferred embodiment, the amino acids residues of the synthetic CDR1 and CDR2 are determined by the following rules:

at CDR1 position 1: Y, R, S, T, F, G, A, or D;
at CDR1 position 2: Y, S, F, G or T;
at CDR1 position 3: Y, S, F, or W;
at CDR1 position 4: Y, R, S, T, F, G, A, W, D, E, K or N;
at CDR1 position 5: S, T, F, G, A, W, D, E, N, I, H, R, Q, or L;
at CDR1 position 6: S, T, Y, D, or E;
at CDR1 position 7: S, T, G, A, D, E, N, I, or V;
at CDR2 position 1: R, S, F, G, A, W, D, E, or Y;
at CDR2 position 2: S, T, F, G, A, W, D, E, N, H, R, Q, L or Y;
at CDR2 position 3: S, T, F, G, A, W, D, E, N, H, Q, P;
at CDR2 position 4: G, S, T, N, or D;
at CDR2 position 5: S, T, F, G, A, Y, D, E, N, I, H, R, Q, L, P, V, W, K or M;
at CDR2 position 6: S, T, F, G, A, Y, D, E, N, I, H, R, Q, L, P, V, W, or K;
at CDR2 position 7: S, T, F, G, A, Y, D, E, N, I, H, R, Q, L, P, or V;

In one related embodiment that may be combined with the preceding embodiment, said CDR3 amino acid sequence comprises between 9 and 18 amino acids. In one related embodiment that may be combined with the preceding embodiment, said CDR3 amino acid sequence comprises amino acid residues selected among one or more of the following amino acids: S, T, F, G, A, Y, D, E, N, I, H, R, Q, L, P, V, W, K, M.

The invention also relates to a synthetic single domain antibody library obtainable by the method described above and comprising at least 3·10⁹ distinct single domain antibody coding sequences.

The invention further concerns the use of said synthetic single domain antibody library, in a screening method, e.g. phage display, for identifying a synthetic single domain antibody that binds to a target of interest, for example a human protein.

Finally, the invention deals with an antigen-binding protein, comprising a synthetic single domain antibody of the following formula: FRW1-CDR1-FRW2-CDR2-FRW3-CDR3-FRW4, wherein said framework regions FRW1, FRW2, FRW3, and FRW4 contains at least the following amino acid residues FRW2-V4/, FRW2-G11, FRW2-L12 and FRW2-W14, and optionally one or more of the following amino acid residues
FRW1-V5, FRW1-E6, FRW1-L11, FRW3-V35, FRW4-R2, FRW4-L7, and/or
FRW1-P14, FRW3-S17, FRW3-R29, FRW3-A30, FRW2-S16, FRW3-K18, FRW3-V21, FRW3-Y22, FRW3-L23, FRW3-S27

In some embodiments, the antigen binding protein comprises at least one the following amino acid residues FRW2-V4/, FRW2-G11, FRW2-L12 and FRW2-W14, and optionally one or more of the following amino acid residues: FRW1-V5, FRW1-E6, FRW1-L11, FRW3-V35, FRW4-R2, FRW4-L7, FRW1-P14, FRW3-S17, FRW3-R29, FRW3-A30, FRW2-S16, FRW3-K18, FRW3-V21, FRW3-Y22, FRW3-L23, FRW3-S27.

In some embodiments, the antigen binding protein amino acid sequence contains the following amino acid residues FRW2-V4, FRW2-G11, FRW2-L12, FRW2-W14, FRW1-V5, FRW1-E6, FRW1-L11, FRW3-V35, FRW4-R2, FRW4-L7, FRW1-P14, FRW3-S17, FRW3-R29, FRW3-A30, FRW2-S16, FRW3-K18, FRW3-V21, FRW3-Y22, FRW3-L23, FRW3-S27.

In some of these embodiments, the antigen binding protein contains at least the amino acid residues consisting of FRW2-V4, FRW2-G11, FRW2-L12, and FRW2-W14. In even more specific embodiments, the scaffold comprises at least one of the amino acid residues from the group consisting of FRW2-V5, FRW3-V21 and FRW4-R2.

In one specific embodiment which may be combined with the preceding embodiments, the antigen-binding protein comprises a synthetic single domain antibody having one or more of the following functional properties:
a) it can be expressed as soluble single domain antibody in E. coli periplasm,
b) it can be expressed as soluble intrabodies in E. coli, yeast or other eukaryote cytosol,
c) it does not aggregate when expressed in mammalian cells, including as a fusion protein (e.g. fluorescent protein fusion).

In some embodiments, the framework regions of the antigen-binding protein are derived from VHH framework regions FRW1, FRW2, FRW3, and FRW4 of Lama species.

In some embodiments, the antigen-binding protein, as above defined has framework regions consisting of FRW1 of SEQ ID NO:1, FRW2 of SEQ ID NO:2, FRW3 of SEQ ID NO:3, and FR4 of SEQ ID NO:4.

In preferred embodiments, which may be combined with the preceding embodiments, the amino acid residues of the synthetic CDR1 and CDR2 are distributed as follows:

at CDR1 position 1: Y, R, S, T, F, G, A, or D;
at CDR1 position 2: Y, S, F, G or T;
at CDR1 position 3: Y, S, S, S, F, or W;
at CDR1 position 4: Y, R, S, T, F, G, A, W, D, E, K or N;
at CDR1 position 5: S, T, F, G, A, W, D, E, N, I, H, R, Q, or L;
at CDR1 position 6: S, T, Y, D, or E;
at CDR1 position 7: S, T, G, A, D, E, N, I, or V;
at CDR2 position 1: R, S, F, G, A, W, D, E, or Y;
at CDR2 position 2: S, T, F, G, A, W, D, E, N, H, R, Q, L or Y;
at CDR2 position 3: S, T, F, G, A, W, D, E, N, H, Q, P;
at CDR2 position 4: G, S, T, N, or D;
at CDR2 position 5: S, T, F, G, A, Y, D, E, N, I, H, R, Q, L, P, V, W, K or M;
at CDR2 position 6: S, T, F, G, A, Y, D, E, N, I, H, R, Q, L, P, V, W, or K;
at CDR2 position 7: S, T, F, G, A, Y, D, E, N, I, H, R, Q, L, P, or V;
and the CDR3 amino acid sequence comprises between 9 and 18 amino acids selected among one or more of the following amino acids: S, T, F, G, A, Y, D, E, N, I, H, R, Q, L, P, V, W, K, M.

### DETAILED DESCRIPTION OF THE INVENTION

In the present description, positions of amino acid residues in synthetic single domain antibodies or their fragments are indicated according to their position (from left to right) in each individual sequence as shown in table 1 below.

TABLE 1

| | | |
|---|---|---|
| SEQ ID | FRW1 | EVQLVESGGGLVQPGGSLRLSCAASG |
| NO: 1 | | |
| SEQ ID | FRW2 | MGWVRQAPGKGLEWVSAIS |
| NO: 2 | | |
| SEQ ID | FRW3 | YYADSVKGRFTISRDNSKNTVYLQMNSLRAEDTAV |
| NO: 3 | | YYCA |
| SEQ ID | FRW4 | YRGQGTLVTVSS |
| NO: 4 | | |

The present invention provides a method of making a synthetic single domain antibody library, said method comprising
i. introducing a diversity of synthetic nucleic acids encoding CDR1, CDR2, and CDR3, between the respective framework coding regions of a synthetic single domain antibody to generate nucleic acids encoding a diversity of synthetic single domain antibodies with the same synthetic single domain antibody scaffold amino acid sequence,
   wherein said synthetic single domain scaffold amino acid sequence contains at least the following amino acid residues FRW2-V4, FRW2-G11, FRW2-L12, FRW2-W14. and optionally further comprising one or more of the following residues FRW1-V5, FRW1-E6, FRW1-L11, FRW3-V35, FRW4-R2, FRW4-L7.

In some embodiments, the synthetic single domain scaffold amino acid sequence contains at least one of the following amino acid residues FRW1-P14, FRW3-S17, FRW3-R29, FRW3-A30, and FRW2-S16.

In some embodiments, the synthetic single domain scaffold amino acid sequence contains at least one of the following amino acid residues FRW3-K18, FRW3-V21, FRW3-Y22, FRW3-L23, FRW3-S27.

In some embodiments, the synthetic single domain scaffold amino acid sequence contains at least one of the following amino acid residues FRW2-V4, FRW2-G11, FRW2-L12, FRW2-W14, FRW1-V5, FRW1-E6, FRW1-L11, FRW3-V35, FRW4-R2, FRW4-L7 and optionally further comprising one or more of the following residues FRW1-P14, FRW3-S17, FRW3-R29, FRW3-A30, FRW2-S16, FRW3-K18, FRW3-V21, FRW3-Y22, FRW3-L23, FRW3-S27.

### The Synthetic Single Domain Antibody Scaffold of the Invention

The present disclosure relates to the identification of unique features in framework regions of single domain antibodies, for obtaining a highly stable single domain antibody scaffold and its use in generating synthetic single domain antibody library, such as synthetic single domain antibody phage display library. The resulting hs2dAb with said unique scaffold are highly stable and have very low risks of immunogenicity. Said resulting hs2dAb also exhibit high solubility and high yield of expression supporting facilitated therapeutic uses.

As a starting material for making the library, a nucleic acid encoding single domain antibody may be provided.

As used herein, the term “single domain antibody” or “Nanobody®” (tradename of Ablynx) refers to an antibody fragment with a molecular weight of only 12-15 kDa, consisting of a single monomeric variable antibody domain derived from a heavy chain. Such single domain antibodies (named VHH) can be found in Camelid mammals and are naturally devoid of light chains. For a general description of single domain antibodies, reference is also made to the prior art cited above, as well as to EP 0 368 684, Ward et al. (Nature 1989 Oct. 12; 341 (6242): 544-6), Holt et al, Trends Biotechnol, 2003, 21(11):484-490; and WO 06/030220, WO 06/003388.

In some embodiments, said single domain antibody may derive from:
fragment of natural occurring antibodies devoid of light chains, such as so called VHH antibodies derived from camelid antibodies or so called VNAR fragments derived from shark species antibody, or
human antibodies;
   with amino acid residues: FRW2-V4, FRW2-G11, FRW2-L12, FRW2-W14 and optionally at least one amino acid residue selected from the group consisting of:
, FRW1-V5, FRW1-E6, FRW1-L11, FRW3-V35, FRW4-R2, FRW4-L7; and/or
FRW1-P14, FRW3-S17, FRW3-R29, FRW3-A30, FRW2-S16, FRW3-K18, FRW3-V21, FRW3-Y22, FRW3-L23, FRW3-S27, notably the combination FRW1-P14, FRW2-S16, FRW3-S17, FRW3-R29, and FRW3-A30.

Single domain antibody thus contains at least 4 framework regions interspaced by 3 hypervariable CDR regions, resulting in the following typical antibody variable domain structure: FRW1-CDR1-FRW2-CDR2-FRW3-CDR3-FRW4. Said single domain does not need to interact with light chain antibody variable region to form conventional heterodimer of heavy and light chains antigen-binding antibody structure and be active.

As used herein, the term “synthetic” means that such antibody has not been obtained from fragments of naturally occurring antibodies but produced from recombinant nucleic acids comprising artificial coding sequences.

In particular, the synthetic single domain antibody libraries of the invention have been generated by synthesis of artificial framework and CDR coding sequences. As opposed to libraries obtained by amplification of naive repertoire from non-immunized llama animals, the synthetic single domain antibody library of the invention does not contain mixture of framework and in particular mixture of VHH and conventional VH antibody.

Advantageously, in one preferred embodiment of the synthetic single domain antibody library of the present invention, all single domain antibody clones contain the same framework regions, thereby providing a unique synthetic single domain antibody scaffold.

As used herein, the term “scaffold” refers to the 4 framework regions of the synthetic single domain antibodies of the library of the invention. Typically, all single domain antibodies of a library of the invention have the same scaffold amino acid sequences while their CDRs may be different (i.e.: the diversity of each library is only in the CDR regions).

The synthetic single domain antibody scaffold according to the present invention contains amino acid residues: FRW2-V4, FRW2-G11, FRW2-L12, FRW2-W14 and optionally at least one amino acid residue selected from the group consisting of
FRW1-V5, FRW1-E6, FRW1-L11, FRW3-V35, FRW4-R2, FRW4-L7; and/or
FRW1-P14, FRW3-S17, FRW3-R29, FRW3-A30, FRW2-S16, FRW3-K18, FRW3-V21, FRW3-Y22, FRW3-L23, FRW3-S27, notably FRW1-P14, FRW2-S16, FRW3-S17, FRW3-R29, and FRW3-A30.

In some embodiments, the synthetic single domain scaffold amino acid sequence contains the following amino acid residues FRW2-V4, FRW2-G11, FRW2-L12, FRW2-W14, FRW1-V5, FRW1-E6, FRW1-L11, FRW3-V35, FRW4-R2, FRW4-L7, FRW1-P14, FRW3-S17, FRW3-R29, FRW3-A30, FRW2-S16, FRW3-K18, FRW3-V21, FRW3-Y22, FRW3-L23, FRW3-S27.

Such unique features provide highly stable synthetic single domain antibody with low risk of immunogenicity.

In a specific embodiment, the synthetic single domain antibody scaffold comprises the following framework regions consisting of FRW1 of SEQ ID NO: 1, FRW2 of SEQ ID NO:2, FRW3 of SEQ ID NO: 3 and FRW4 of SEQ ID NO:4, or functional variant framework regions, for example with no more than 1, 2 or 3 conservative amino acid substitutions within each framework region, more preferably, within only one framework region.

In some of these embodiments, the synthetic single domain antibody scaffold contains at least the amino acid residues consisting of FRW2-V4, FRW2-G11, FRW2-L12, and FRW2-W14. In even more specific embodiments, the scaffold comprises at least one of the amino acid residues from the group consisting of FRW2-V5, FRW3-V21 and FRW4-R2. As previously mentioned these amino acids residues at the indicated positions allow to obtain singles domain antibodies with reduced immunogenicity (notably for the FR2V5 residue) as well as to improve their thermal stability, solubility and bioproduction (in particular for the FRW4-E2 residue).

Conservative amino acid substitutions are ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine).

In another embodiment, the synthetic single domain antibody scaffold comprises functional variants of FRW1, FRW2, FRW3 and FRW4 framework regions having at least 90%, preferably 95% or 99% identity to SEQ ID NOs 1-4 respectively. Typically, amino acid residues FRW2-V4, FRW2-G11, FRW2-L12 and FRW2-W14 are preserved.

As used herein, the percent identity between two sequences is a function of the number of identical positions shared by the sequences (i.e., % identity=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Myers and W. Miller (Comput. Appl. Biosci. 4: 11-17, 1988) which has been incorporated into the ALIGN program. In addition, the percent identity between two amino acid sequences can be determined using the Needleman and Wunsch (J. Mol. Biol. 48:443-453, 1970) algorithm which has been incorporated into the GAP program in the GCG software package. Yet another program to determine percent identity is CLUSTAL (M. Larkin et al., Bioinformatics 23:2947-2948, 2007; first described by D. Higgins and P. Sharp, Gene 73:237-244, 1988) which is available as stand-alone program or via web servers (see http://www.clustal.org/).

Functional variants may be tested for their capacity to retain the advantageous properties of said synthetic single domain scaffold of the present invention. In particular, they may be tested for their capacity to retain at least one or more of the following properties:
i. it can be expressed as soluble single domain antibody in E. coli periplasm,
ii. it can be expressed as soluble intrabodies in E. coli, yeast or other eukaryote cytosol,
iii. it does not aggregate when expressed in mammalian cells, including as a fusion proteins (e.g. fluorescent protein fusion).

Assays for testing the above properties are described in the Examples.

For example, a reference synthetic single domain antibody coding sequence can be constructed by grafting reference CDRs coding sequences (such as the CDRs of clone F8 of SEQ ID NO: 9) into a variant scaffold coding sequence to be tested (with homologous sequences to SEQ ID NOs 1-4). This reference synthetic single domain antibody coding sequence allows to produce a reference synthetic single domain antibody which can be assayed for the above properties.

### Introduction of CDR Diversity in the Selected Single Domain Antibody Scaffold

Methods for generating CDRs diversity for antibody libraries, in particular by random, or directed, synthesis of CDR coding sequences and cloning into corresponding framework sequences have been widely described in the art.

The synthetic single domain antibody libraries of the present invention are generated similarly by introducing CDR high diversity into the unique selected scaffold sequence, for example, as described in Lindner, T., H. Kolmar, U. Haberkorn, and W. Mier. 2011. Molecules. 16: 1625-1641.

In one preferred embodiment of the present invention, the position of each amino acid sequence of synthetic CDR1 and CDR2 is rationally designed to mimic natural diversity of CDRs in human repertoire.

Cysteines are voluntarily avoided because of their thiol groups which may interfere with intracellular expression and functionality. Besides, arginine and hydrophobic residues may also be avoided because of the high-risk aggregation of the resulting antibody. A low proline rate is also preferred because it provides more flexibility in the CDRs. Preferably, serine, threonine and tyrosine are the most frequent residues in all three CDRs, as being involved in bonds with the epitope. Aspartate and glutamate may also be enriched at some positions in order to increase solubility. For CDR3 sequences, the lengths may influence the binding potential to different epitope shape, in particular cavity. Therefore, different lengths of CDR3 sequences may be introduced into the libraries.

In one specific embodiment, the skilled person may select the amino acid residues of the synthetic CDR1 and CDR2 according to the following rules:

at CDR1 position 1: Y, R, S, T, F, G, A, or D;
at CDR1 position 2: Y, S, F, G or T;
at CDR1 position 3: Y, S, F, or W;
at CDR1 position 4: Y, R, S, T, F, G, A, W, D, E, K or N;
at CDR1 position 5: S, T, F, G, A, W, D, E, N, I, H, R, Q, or L;
at CDR1 position 6: S, T, Y, D, or E;
at CDR1 position 7: S, T, G, A, D, E, N, I, or V;
at CDR2 position 1: R, S, F, G, A, W, D, E, or Y;
at CDR2 position 2: S, T, F, G, A, W, D, E, N, H, R, Q, L or Y;
at CDR2 position 3: S, T, F, G, A, W, D, E, N, H, Q, P;
at CDR2 position 4: G, S, T, N, or D;
at CDR2 position 5: S, T, F, G, A, Y, D, E, N, I, H, R, Q, L, P, V, W, K or M;
at CDR2 position 6: S, T, F, G, A, Y, D, E, N, I, H, R, Q, L, P, V, W, or K;
at CDR2 position 7: S, T, F, G, A, Y, D, E, N, I, H, R, Q, L, P, or V;

Furthermore, in another specific embodiment, CDR3 amino acid sequence comprises between 9 and 18 amino acids selected among one or more of the following amino acids: S, T, F, G, A, Y, D, E, N, I, H, R, Q, L, P, V, W, K, M.

The above rules of occurrence are used as a guidance for generating preferred libraries of the invention, however, other libraries with different occurrence rules are also part of the invention, as long as they contain the advantageous synthetic single domain antibody scaffold of the present invention.

In specific embodiments, only a significant proportion of the clones of the library may follow strictly the above rules of occurrence. For example, statistically, at least 50%, 60%, 70%, 80% or at least 90% of the clones of the library follow the above rules of occurrence of amino acid residues in CDR1, CDR2 and CDR3 positions.

In order to respect these occurrences of amino acid positions, and to avoid the occurrence of in frame stop, or cysteine or reduce frameshift, advanced gene synthesis approaches are preferably used. These methods encompass, but are not limited to, double strand DNA triple blocks as described in Van den Brulle et al., 2008, Biotechniques 45(3): 340-3, tri-nucleotide synthesis, or other codon-controlled and more generally position-controlled degenerate synthesis approaches.

In specific embodiments, codon bias may further be optimized for example for host cell species, for example, mammalian host cells expression, using well known methods.

In one specific embodiment, the coding sequence is designed so that it does not contain undesired restriction sites, for example, restriction sites that are used for cloning the coding sequence into the appropriate cloning or expression vector.

The resulting diverse coding sequences are introduced into a suitable expression or cloning vectors for antibody libraries. In a specific embodiment, the expression vector is a plasmid. In another preferred embodiment, the expression vector is suitable for generating phage display libraries. Two different types of vectors may be used for generating phage display libraries: phagemid vectors and phage vectors.

Phagemids are derived from filamentous phage (Ff-phage-derived) vectors, containing the replication origin of a plasmid. The basic components of a phagemid mainly include the replication origin of a plasmid, the selective marker, the intergenic region (IG region, usually contains the packing sequence and replication origin of minus and plus strands), a gene of a phage coat protein, restriction enzyme recognition sites, a promoter and a DNA segment encoding a signal peptide. Additionally, a molecular tag can be included to facilitate screening of phagemid-based library. Phagemids can be converted to filamentous phage particles with the same morphology as Ff phage by co-infection with the helper phages, such as R408, M13K07 and VCSM13 (Stratagene). One example of phage vector is fd-tet (Zacher et al, gene, 1980, 9, 127-140) which consists of fd-phage genome and a segment of TnlO inserted near the phage genome origin of replication. Examples of promoters for use in phagemid vectors include, without limitation, PlacZ or PT7, examples of signal peptide include without limitation pelB leader, gill, CAT leader, SRP or OmpA signal peptide.

Other phage-display methods use lytic phages like T4 or T7. Vectors other than phages may also be used to generate display libraries, including vectors for bacterial cell display (Daugherty et al., 1999 Protein Eng. July; 12(7):613-21, Georgiou et al., 1997 Nat Biotechnol. 1997 January; 15(1):29-34), yeast cell display (Boder and Wittrup, Nat Biotechnol. 1997 June; 15(6):553-7) or ribosome display (Zahnd C, Amstutz P, Pluckthun A. Nat Methods. 2007 March; 4(3):269-79). DNA display (Eldridge et al., Protein Engineering, Design & Selection vol. 22 no. 11 pp. 691-698, 2009) and surface display on mammalian cells (Rode H J, et al. Biotechniques. 1996 October; 21(4):650, 652-3, 655-6, 658) have also been reported. Non display methods like yeast two-hybrid may also be used to select relevant binders from the library (Visintin et al., 1999 Proc Natl Acad Sci USA 96, 1 1723-1 1728).

In one preferred embodiment, in order to avoid generating empty vectors, positive selection of recombinant coding sequence in the cloning vectors bearing a suicide gene is applied (see for example Philippe Bernard, 1996, BioTechniques, Vol 21, No 2 “Positive Selection of Recombinant DNA by CcdB”).

Preferably, the theoretical diversity as calculated by all possible combination of CDR amino acid residues as designed for generating the antibody library is at least 10¹¹ or at least 10¹², notably 10²³ unique sequences.

Synthetic Single Domain Antibody Library of the Invention and their Use

Consequently, according to another aspect, the invention relates to a synthetic single domain antibody library obtainable or obtained by the previous method.

As used herein, the term “synthetic single domain antibody library” thus encompasses nucleic acid libraries comprising said synthetic single domain antibody coding sequences with high diversity, optionally included in a cloning vector or expression vector. The term “synthetic single domain antibody library” further includes any transformed host cells or organisms, with said nucleic acid libraries, and more specifically, bacterial, yeast or filamentous fungi, or mammalian cells transformed with said nucleic acid libraries, or bacteriophages or viruses containing said nucleic acid libraries. The term “synthetic single domain antibody library” further includes the corresponding mixture of diverse antibodies encoded by said nucleic acid library. As used herein, the term “clone” will refer to each unique individual of the antibody library, whether, nucleic acids, host cells, or single domain antibodies.

In one specific embodiment of the invention, the synthetic single domain antibody library of the present invention comprises at least 1×10⁸, notably 1.6×10⁹ diverse clones.

This library may be used in a screening method, for identifying a synthetic single domain antibody that binds specifically to a target of interest. Any known screening methods for identifying binders with specific affinity to a target of interest may be used with the synthetic single domain antibody libraries of the invention. Such methods include without limitation phage display technologies, bacterial cell display, yeast cell display, mammalian cell display or ribosome display.

Preferably, the screening method is the phage display.

Preferably, the target of interest is a therapeutic target, and the synthetic single domain antibody library is used to identify synthetic single domain antibody with specific binding to said therapeutic target. In specific embodiments, the target of interest comprises at least an antigenic determinant. In specific embodiments, the target is a saccharide or polysaccharide, a protein or glycoprotein, a lipid. In one specific embodiment, said target of interest is of plant, yeast, fungus, insect, mammalian or other eukaryote cell origins. In another specific embodiment, said target of interest is of bacterial, protozoan or viral origin.

In one specific embodiment, “a single domain antibody that binds specifically to a target of interest” is intended to refer to single domain antibody that binds to the target of interest with a K_{D} of 1 mM or less, 100 μM or less, 10 μM, 1 μM, 100 nM, 10 nM, 1 nM, 100 pM, 10 pM or less. This does not exclude that said single domain antibody also binds to other antigens.

The term “K_{D}”, as used herein, is intended to refer to the dissociation constant, which is obtained from the ratio of K_{d} to Kₐ (i.e. K_{d}/Kₐ) and is expressed as a molar concentration⁻¹(M⁻¹). K_{D} values for antibodies can be determined using methods well established in the art. A method for determining the K_{D} of an antibody is by using surface plasmon resonance, or using a biosensor system such as a Biacore® system or Proteon®.

### Antigen-Binding Protein of the Invention

Considering the high diversity of the synthetic single domain antibody libraries of the invention, the skilled person can obtain synthetic single domain antibody with high affinity and high specificity to a target of interest, by conventional screening methods, such a phage display.

The resulting synthetic single domain antibody can then be further modified for generating appropriate antigen-binding protein. In particular, the CDR residues may be modified for example to increase the antibody affinity to the target of interest, improve its folding or its production, using technologies known in the art (mutagenesis, affinity maturation).

Accordingly, another aspect of the invention further relates to an antigen-binding protein, comprising a synthetic single domain antibody of the following formula: FRW1-CDR1-FRW2-CDR2-FRW3-CDR3-FRW4, wherein said framework regions FRW1, FRW2, FRW3, and FRW4 contains the following amino acids residues: FRW2-V4, FRW2-G11, FRW2-L12, FRW2-W14 and optionally at least one amino acid residue selected from the group consisting of
FRW1-E6, FRW1-L11, FRW3-V35, FRW4-R2 and FRW4-L7; and/or
FRW1-P14, FRW3-S17, FRW3-R29, FRW3-A30, FRW2-S16, FRW3-K18, FRW3-V21, FRW3-Y22, FRW3-L23, FRW3-S27, and notably FRW1-P14, FRW2-S16, FRW3-S17, FRW3-R29, and FRW3-A30.

In some embodiments, said synthetic single domain scaffold of the present disclosure comprise amino acid residues FRW1-V5, FRW1-E6, FRW1-L11, FRW3-V35, FRW4-R2, and FRW4-L7 and/or FRW1-P14, FRW3-S17, FRW3-R29, FRW3-A30, FRW2-S16, FRW3-K18, FRW3-V21, FRW3-Y22, FRW3-L23, FRW3-S27, notably FRW1-P14, FRW2-S16, FRW3-S17, FRW3-R29, and FRW3-A30.

In some embodiments, the synthetic single domain scaffold amino acid sequence contains the following amino acid residues FRW2-V4, FRW2-G11, FRW2-L12, FRW2-W14, FRW1-V5, FRW1-E6, FRW1-L11, FRW3-V35, FRW4-R2, FRW4-L7, FRW1-P14, FRW3-S17, FRW3-R29, FRW3-A30, FRW2-S16, FRW3-K18, FRW3-V21, FRW3-Y22, FRW3-L23, FRW3-S27.

In some embodiments, the framework regions are derived from VHH framework regions FRW1, FRW2, FRW3, and FRW4 of Lama species.

In one preferred embodiment, the synthetic single domain antibody comprises either of the following features:

(i) framework regions FRW1 of SEQ ID NO: 1, FRW2 of SEQ ID NO:2, FRW3 of SEQ ID NO:3, and FRW4 of SEQ ID NO:4,
(ii) functional variant framework regions having no more 1, 2 or 3 amino acid conservative substitutions and retaining advantageous synthetic single domain properties,
(iii) functional variant framework regions FRW1, FRW2, FRW3 and FRW4 having at least 60, 70, 80, 90, 95, 96, 97, 98, or 99 percent sequence identity to SEQ ID NOs: 1-4 respectively and retaining advantageous synthetic single domain properties,

Typically, one or more amino acid residues within the framework regions can be replaced with other amino acid residues from the same side chain family, and the new polypeptide variant can be tested for retained advantageous properties using the functional assays described herein.

Such advantageous properties are one or more of the following properties:
i. It can be expressed as soluble single domain antibody in E. coli periplasm.

No aggregation is observed upon expression, extraction and purification from the periplasm when using simple centrifugation analysis. Typically, a yield exceeding 1 mg/L with a pelB leader peptide may be preferably obtained in E. coli strains.
ii. It can be expressed as soluble intrabodies in E. coli cytosol

No aggregation is observed upon expression, extraction and purification from the periplasm when using simple centrifugation analysis. For example, antibodies may be expressed in E. coli strains BL21(DE3) at a yield exceeding 50 mg/liter with a T7 promoter.
iii. It does not aggregate when expressed in mammalian cell lines as fluorescent protein fusions.

Preferably, no aggregation should be detected when the antigen-binding protein containing the synthetic single domain antibody is expressed as fluorescent protein fusion. Analysis can be done using simple fluorescence imaging.

Preferably, the amino acid residues of the synthetic CDR1 and CDR2 may be:

at CDR1 position 1: Y, R, S, T, F, G, A, or D;
at CDR1 position 2: Y, S, F, G or T;
at CDR1 position 3: Y, S, S, S, F, or W;
at CDR1 position 4: Y, R, S, T, F, G, A, W, D, E, K or N;
at CDR1 position 5: S, T, F, G, A, W, D, E, N, I, H, R, Q, or L;
at CDR1 position 6: S, T, Y, D, or E;
at CDR1 position 7: S, T, G, A, D, E, N, I, or V;
at CDR2 position 1: R, S, F, G, A, W, D, E, or Y;
at CDR2 position 2: S, T, F, G, A, W, D, E, N, H, R, Q, L or Y;
at CDR2 position 3: S, T, F, G, A, W, D, E, N, H, Q, P;
at CDR2 position 4: G, S, T, N, or D;
at CDR2 position 5: S, T, F, G, A, Y, D, E, N, I, H, R, Q, L, P, V, W, K or M;
at CDR2 position 6: S, T, F, G, A, Y, D, E, N, I, H, R, Q, L, P, V, W, or K;
at CDR2 position 7: S, T, F, G, A, Y, D, E, N, I, H, R, Q, L, P, or V;
and CDR3 amino acid sequence comprises between 9 and 18 amino acids selected among one or more of the following amino acids: S, T, F, G, A, Y, D, E, N, I, H, R, Q, L, P, V, W, K, M.

Accordingly, in one preferred embodiment, the antigen-binding protein of the invention, essentially consists of a synthetic single domain antibody of the general formula FRW1-CDR1-FRW2-CDR2-FRW3-CDR3-FRW4.

In such embodiment, more preferably, FRW1 is SEQ ID NO: 1, or a functional variant of SEQ ID NO: 1 with 1, 2 or 3 amino acid substitutions, FRW2 is SEQ ID NO:2, or a functional variant of SEQ ID NO:2 with 1, 2 or 3 amino acid substitutions; FRW3 is SEQ ID NO:3, or a functional variant of SEQ ID NO:3 with 1, 2 or 3 amino acid substitutions; FRW4 is SEQ ID NO:4, or a functional variant of SEQ ID NO:4 with 1, 2 or 3 amino acid substitutions; CDR1, CDR2 amino acid sequences have amino acid residues as follows:

at CDR1 position 1: Y, R, S, T, F, G, A, or D;
at CDR1 position 2: Y, S, F, G or T;
at CDR1 position 3: Y, S, S, S, F, or W;
at CDR1 position 4: Y, R, S, T, F, G, A, W, D, E, K or N;
at CDR1 position 5: S, T, F, G, A, W, D, E, N, I, H, R, Q, or L;
at CDR1 position 6: S, T, Y, D, or E;
at CDR1 position 7: S, T, G, A, D, E, N, I, or V;
at CDR2 position 1: R, S, F, G, A, W, D, E, or Y;
at CDR2 position 2: S, T, F, G, A, W, D, E, N, H, R, Q, L or Y;
at CDR2 position 3: S, T, F, G, A, W, D, E, N, H, Q, P;
at CDR2 position 4: G, S, T, N, or D;
at CDR2 position 5: S, T, F, G, A, Y, D, E, N, I, H, R, Q, L, P, V, W, K or M;
at CDR2 position 6: S, T, F, G, A, Y, D, E, N, I, H, R, Q, L, P, V, W, or K;
at CDR2 position 7: S, T, F, G, A, Y, D, E, N, I, H, R, Q, L, P, or V;
and CDR3 amino acid sequence comprises between 9 and 18 amino acids selected among one or more of the following amino acids: S, T, F, G, A, Y, D, E, N, I, H, R, Q, L, P, V, W, K, M.

Another aspect of the invention pertains to nucleic acid molecules that encode the antigen-binding proteins of the invention. The invention thus provides an isolated nucleic acid encoding at least said synthetic single domain antibody portion of the antigen-binding protein.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g. other cellular nucleic acids or proteins, by standard techniques, including alkaline/SDS treatment, CsCI banding, column chromatography, agarose gel electrophoresis and others well known in the art. See, F. Ausubel, of al., ed. 1987 Current Protocols in Molecular Biology, Greene Publishing and Wiley Interscience, New York. A nucleic acid of the invention can be, for example, DNA or RNA and may or may not contain intronic sequences. In an embodiment, the nucleic acid is a DNA molecule. The nucleic acid may be present in a vector such as a phage display vector, or in a recombinant plasmid vector. In one specific embodiment, the invention thus provides an isolated nucleic acid or a cloning or expression vector comprising at least one or more of the following nucleic acid sequences: SEQ ID NO:5, SEQ ID NO:6, SEQ ID NO:7, SEQ ID NO:8, encoding respectively framework regions FRW1, FRW2, FRW3 and FRW4 of SEQ ID NOs 1-4, or variant corresponding sequences with at least 90% identity to said SEQ ID NOs 5-8, encoding functional variants of FRW1, FRW2, FRW3, and FRW4 of SEQ ID NOs 1-4.

DNA fragments encoding the antigen-binding proteins, as described above and in the Examples, can be further manipulated by standard recombinant DNA techniques, for example to include any signal sequence for appropriate secretion in expression system, any purification tag and cleavable tag for further purification steps. In these manipulations, a DNA fragment is operatively linked to another DNA molecule, or to a fragment encoding another protein, such as a purification/secretion tag or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The antigen-binding proteins of the invention can be produced in a host cell transfectoma using, for example, a combination of recombinant DNA techniques and gene transfection methods as is well known in the art. For expressing and producing recombinant antigen-binding proteins of the invention in host cell transfectoma, the skilled person can advantageously use its own general knowledge related to the expression and recombinant production of antibody molecules or single domain antibody molecules.

The invention thus provides a recombinant host cell suitable for the production of said antigen-binding proteins of the invention, comprising the nucleic acids, and optionally, secretion signals. In a preferred aspect the host cell of the invention is a mammalian cell line. The invention further provides a process for the production of an antigen-binding protein, as described previously, comprising culturing the host cell under appropriate conditions for the production of the antigen-binding protein, and isolating said protein.

Mammalian host cells for secreting the antigen-binding proteins of the invention, include CHO, such as dhfr-CHO cells, (described by Urlaub and Chasin, 1980, Proc. Natl. Acad. Sci. USA 77:4216-4220) used with a DHFR selectable marker, e.g. as described in R. J. Kaufman and P. A. Sharp, 1982 Mol. Biol. 159:601-621, NSO myeloma cells, or the pFuse expression system from Invivogen, as described in Moutel, S., El Marjou, A., Vielemeyer, O., Nizak, C, Benaroch, P., Dubel, S., and Perez, F. (2009). A multi-Fc-species system for recombinant antibody production. BMC Biotechnol 9, 14, COS cells and SP2 cells or human cell lines (including PER-C6 cell lines, Crucell or HEK293 cells, Yves Durocher et al., 2002, Nucleic acids research vol. 30, No 2 p 9). When said nucleic acids encoding antigen-binding proteins of the invention are introduced into mammalian host cells, the antigen-binding proteins are produced by culturing the host cells for a period of time sufficient to allow for expression of the recombinant polypeptides in the host cells or secretion of the recombinant polypeptides into the culture medium in which the host cells are grown and proper refolding to produce said antigen-binding proteins.

The antigen-binding protein can then be recovered from the culture medium using standard protein purification methods.

In one specific embodiment, the present invention provides multivalent antigen-binding proteins of the invention, for example in the form of a complex, comprising at least two identical or different synthetic single domain antibody amino acid sequences of the invention. In one embodiment, the multivalent protein comprises at least two, three or four synthetic single domain antibody amino acid sequences. The synthetic single domain amino acid sequences can be linked together via protein fusion or covalent or non-covalent linkages.

In another aspect, the present invention provides a composition, e.g. a pharmaceutical composition, containing one or a combination of the antigen-binding proteins of the present invention, formulated together with one or more pharmaceutically acceptable vehicles or carriers.

Pharmaceutical formulations of the invention may be prepared for storage by mixing the proteins having the desired degree of purity with optional physiologically acceptable carriers, excipients or stabilizers (Remington: The Science and Practice of Pharmacy 20th edition (2000)), in the form of aqueous solutions, lyophilized or other dried formulations.

Examples of suitable aqueous and non-aqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage.

In the following, the invention will be illustrated by means of the following examples and FIGURES.

### FIGURES LEGENDS

FIG. 1. (A). Affinity determination of nanobodies to recombinant protein via surface plasmon resonance spectroscopy. Single cycle kinetics analysis was performed on immobilized FGFR4 through covalent amine binding on the dextran based sensor chip. The analytes F8 and mCh were injected in 5 different concentrations followed by a dissociation phase. A final dissociation step was added after the last injection step to determine Koff rates for the KD calculations. The black curves represent the measured data and red curves show the fit analysis (heterogeneous ligand model) performed with the BIAevaluation software.

### EXAMPLES

Validation of the Fully Humanized s2dAb Scaffold

Validation of the scaffold was done using CDR grafting. CDRs from VHH antibodies were inserted into the fully humanized single domain scaffold as herein described. Antibodies targeting various antigens (GFP, mCherry, alpha-tubulin, MUC18) were inserted and the resulting sdAbs used to check that these fully human sdAbs behave as their parental VHH counterparts in terms of antigen detection, display at the phage surface, expression in the bacteria periplasm, expression in the bacteria cytosol, expression in mammalian cell cytosol. Despite the absence of camelid-specific amino acids thought to be essential for stability and solidity, this showed that this fully human sdAb enable efficient production and stability in reducing environment.

### Building a Synthetic Phage Display Library Based on the Design Disclosed Herein

Several ways exist to build synthetic and diverse library and we used here an oligonucleotide-based approach (provided by Twist Bioscience). Synthetic genes based on the design describe here were ordered and inserted in a modified pHEN2 plasmid bearing 3 myc tags. A library of 1.6 10⁹ clones, the Gimli-1 library, was constructed.

Use of Fully Humanized sdAb

Examples of fully humanized sdAb (resulting from CDR grafting or from selection from the Gimli-1 library) were tested to validate the use of the design disclosed here for various antibody-based applications like immunostaining, inhibition of signal transduction or cell targeting, including CAR-T cell development. Fully human sdAb were used as monomeric soluble forms, displayed on phages, fused to Fc domains or fused to CAR-T scaffolds.

### Phage Display Selection of GFP-Specific Nanobodies

A screening in native conditions was performed using the GFP protein as a target. Four non redundant clones out of 80 analysed detected GFP-Rabb, by immunofluorescence. Importantly, we observed that these antibodies were usable as intrabodies against recombinant GFP expressed in Hela cells (see for example the anti_GFP_Gimli_D8 of SEQ ID NO:10).

### Phage Display Selection of Tubulin Nanobodies

A screening was performed in native condition (Nizak, 2005, see supra) using biotinylated tubulin (Cytoskeleton) as a target. After three rounds of selection, 80 clones were screened at random by immunofluorescence on HeLa cells fixed with methanol. 71 recombinant Ab stained the endogenous tubulin (34 unique sequences) (see for example the anti_tubulin_Gimli_B1 of SEQ ID NO:11).

### Phage Display Selection of FGFR4-Specific Nanobodies

Identification for antibodies targeting the cell surface of cancer cells were exemplified by screening for FGFR4-targeting sdAb. The screening of FGFR4-binding nanobodies was performed using the fully humanized sdAb library Gimli-1. We performed a phage display selection with three rounds of biopanning against recombinant FGFR4. In order to verify the binding specificity for FGFR4, we used FGFR4 knocked-out cells RMS cells (from M. Bernasconi, University of Zurich), and tested 80 phage clones the screening for their binding to Rh4 FGFR4 wildtype cells (Rh4-FR4 wt) and Rh4 FGFR4 knockout cells (Rh4-FR4ko). Flow cytometry analysis revealed 55 phage clones from Gimli-1 library binding to the Rh4-FR4 wt cells only. Sanger sequencing of the 55 phage clones revealed that 28 unique nanobodies from the Gimli-1 library were obtained. Next, phage clones from the Gimli library (i.e. Gimli-1: A4, F8, F11, H2) that showed the best binding to Rh4-FR4 wt by flow cytometry were expressed recombinantly. As negative control, we expressed an anti-mCherry nanobody (mCh). Recombinant nanobodies of approximately 17 kDa were engineered to be expressed with a C-terminal Myc/6×His-tag and an additional cysteine for maleimide coupling. 6×His-tag purification and size exclusion chromatography resulted in proteins of high purity, with yields in the range of 3-16 mg per liter of bacterial culture.

Selected nanobodies bind to FGFR4-expressing cells Validation of the binding of recombinant nanobodies to cell-surface expressed FGFR4 was performed with Rh4-FR4 wt and Rh4-FRK4ko cells by flow cytometry. A FITC-labeled anti-6×His-tag antibody was used to detect surface-bound nanobodies. Three of the recombinant nanobodies tested displayed no significant binding to Rh4-FR4 wt cells (A4, F11, H2, data not shown) whereas recombinant nanobody F8 (SEQ ID NO:9) showed a specific binding to Rh4-FR4 wt cells and no binding to Rh4-FR4ko cells. As expected, the anti-mCherry negative control nanobody did not bind to Rh4-FR4 wt nor to Rh4-FR4ko cells. Median fluorescence intensities (MFIs) of the the FGFR4 binder incubated with Rh4-FR4 wt cells were in the range of 400, but anti-mCherry negative control, or the anti-6×His-tag antibody only displayed MFI of 200, similar to the binding to Rh4-FR4ko cells.

### Nanobodies High Affinity Binding to FGFR4

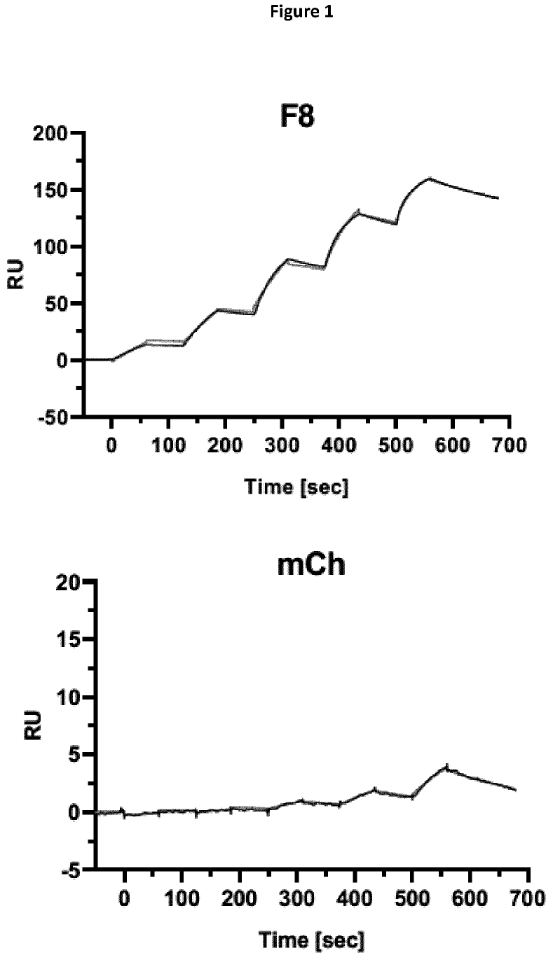

To determine the binding affinity of the nanobody to FGFR4, we performed surface plasmon resonance (SPR) spectroscopy with recombinant FGFR4. As already mentioned above, FGFR1 and FGFR2 are expressed on Rh4-FR4ko cells and flow cytometry analysis indicated no binding of the nanobody to the cells. To further confirm FGFR4-specificity, we included also affinity measurements with recombinant FGFR1, FGFR2 and FGFR3. Nanobodies F8, and mCh were injected in five different concentrations on a FGFR coated chip (Suppl. table 1). Except for the negative control mCh, calculated K_{D} values for FGFR4 binding were in the nano- and picomolar range (FIG. 1; Table 1). Affinity parameters could not be fitted with a 1:1 binding model and best fits were obtained with the heterogeneous ligand model of the BIA evaluation software resulting in two K_{D} values for each candidate. Measurements of the affinities to the receptor family isoforms FGFR1 and FGFR3 showed as expected no binding of the analytes. The SPR data confirmed the strong binding F8 to FGFR4 and further suggests that F8 has a strict FGFR4 specificity.

TABLE 2
Surface plasmon resonance spectroscopic determination of nanobody binding
affinities to FGFR4. Measured data was fitted with the heterogeneous ligand model and
revealed association-and dissociation constants (kₒₙ and k_{off}) used for calculating affinities in
terms of dissociation equilibrium constants K_{D} (k_{off}/kₒₙ). The maximal analyte binding signal
Rmax is indicated in RU for both determined K_{D} and resembles their fraction within the amount
of total bound nanobodies.

| Nanobody | kₒₙ1(1/M*s) | k_{off}1(1/s) | K_{D}1(M) | kₒₙ2(1/M*s) | k_{off}2(1/s) | K_{D}2(M) | Rₘₐₓ1(RU) | Rₘₐₓ2(RU) |
|---|---|---|---|---|---|---|---|---|
| F8 | 5.45E+04 | 1.04E−06 | 1.91E−11 | 1.35E+06 | 5.57E−03 | 4.14E−09 | 83.0 | 86.4 |
| mCh | 2.60E+03 | 5.11E−03 | 1.96E−06 | 2.32E+03 | 5.05E−03 | 2.18E−06 | 20.7 | 20.7 |

### Materials and Methods

### CDR Grafting

In silico design was done so that CDRs of VHH binding to known targets, for example mCherry (but also GFP, Tubulin or MUC18), were grafted in the scaffold disclosed herein. Synthetic genes were ordered and cloned into pHEN2-derivated plasmid for expression in E. coli and phage display and in fusion to a fluorescent protein for expression in mammalian cytosol.

Soluble Expression in E. coli Periplasm

Single domain antibody fragments can be subcloned in a pHEN2 derivated bacterial periplasm expression vector and expressed downstream of the pelB secretion sequence. Freshly transform colonies can be grown in Terrific Broth medium supplemented with 1% glucose and 100 μg/ml ampicillin antibiotic until A600=0.6-0.8 was reached. The expression of antibody fragment tagged with 6 His can be then induced with 500 μM isopropyl P-D-thiogalactopyranoside for 16 h at 16° C. or 4 h at 28° C. then span down. After centrifugation, the cell pellets can be incubated in Tris-EDTA-Sucrose osmotic shock buffer and centrifuged again. The cell lysates can be cleared and loaded onto an IMAC resin affinity column for poly Histidine tag. The eluted fractions are dialyzed, and the purity of the protein analyzed typically by SDS-PAGE.

Soluble Expression of Intrabodies in E. coli Cytosol

Single domain antibody fragments can be subcloned in a bacterial expression vector under the control of a T7 promoter. The plasmid constructs can be transformed into E. coli BL21(DE3) cells. Single colonies can be grown in LB medium supplemented with 1% glucose and 100 μg/ml ampicillin antibiotic until A600=0.6-0.8 was reached. Antibody fragment expression can then be induced with 500 μM isopropyl β-D-thiogalactopyranoside for 16 h at 16° C. and then be span down. After centrifugation, the cell pellets are lysed and centrifuged again. The cell lysates are cleared and loaded typically onto an IMAC resin affinity column for poly Histidine tag. The eluted fraction is dialyzed, and the purity of the protein analyzed typically by SDS-PAGE.

### Aggregation Assays in Mammalian Cell Expression System

### Functional Expression as Intracellular Antibodies in Eukaryote Cells

Single domain antibody fragments can be subcloned into a mammalian expression vector in order to express it as a fusion with a fluorescent protein and typically under the control of a CMV promoter. Mammalian cell lines are transfected and fluorescence in the cells is observed 24 h or 48 h after transfection.

### Cell Lines

The cell lines Rh4 (kindly provided by Peter Houghton, Research Institute at Nationwide Children's Hospital, Columbus, Ohio), Rh30, HEK293 ft HEK293T (purchased from ATCC, LGC Promochem) were maintained in DMEM supplemented with 10% PBS (both Sigma-Aldrich), 2 mM L-glutamine and 100 U/ml penicillin/streptomycin (both Thermo Fisher Scientific) at 37° C. in 5% CO₂. RMS cell lines were tested and authenticated by cell line typing analysis (STR profiling) in 2014/2015 and positively matched⁴⁸. All cell lines tested negative for mycoplasma.

### Phage Display Selection

Screening for against soluble proteins was performed with biotinylated targets or SBP-tagged targets (e.g. extracellular FGFR4—G&P Biosciences) in native condition (as described in Nizak, C., Moutel, S., Goud, B. & Perez, F. Methods Enzymol. 403, 135-153 (2005)) the herein disclosed single domain antibody library composed of 1.6×10⁹ fully humanized hs2dAb. Briefly, biotinylated antigens or SBP-antigen are diluted to obtain a 10-20 nM (1.5 mL final) and confirm efficient recovery on 50 μL streptavidin-coated magnetic beads (Dynal). As a reference, a solution of 10 nM of a 100-kDa protein represents 1 μg protein/mL (hence per round of selection). One can then compare fractions of bound and unbound samples by Western blot using streptavidin-HRP or anti-AviTag antibodies. For screening, the adequate amount of biotinylated antigen coated beads is incubated for 2 h with the phage library (10¹³ phages diluted in 1 mL of PBS+0.1% Tween 20+2% non-fat milk) Phages were previously adsorbed on empty streptavidin-coated magnetic beads (to remove nonspecific binders). Phage bound to streptavidin-coated beads are recovered on a magnet. 10 times (round 1) or 20 times (round 2 and 3) washes are carried out using PBS+Tween 0.1% on a magnet. Bound phages are eluted using triethylamine (TEA, 100 mM) and eluted phages are neutralized using 1M Tris pH 7.4. Elution are done twice on beads. Eluted phages are then used to infect E. coli (TG1). Note that usually for round 2 and round 3, only 10¹² phages were used as input.

### Protein Expression and Purification

Periplasmic expression of nanobodies was performed in E. coli MC1061 harboring the pSB_init vector enabling protein production with a C-terminal cysteine and 6×His-tag. A 20 ml overnight pre-culture grown in Terrific Broth medium (25 μg/ml Chloramphenicol) was diluted in 2000 ml fresh medium and grown at 37° C. for 2 h. The temperature was then reduced to 25° C. and after 1 h protein expression was induced with 0.02% L-arabinose. The bacterial culture was grown overnight at 25° C. and cells were harvested by centrifugation (12000 g, 15 min) Periplasmic protein extraction was performed with the osmotic shock method. The cells were resuspended with 50 ml lysis buffer 1 (50 mM Tris/HCl, pH 8.0, 20% sucrose, 0.5 mM EDTA, 5 μg/ml lysozyme, 2 mM DTT) and incubated for 30 min on ice. After the addition of ice-cold lysis buffer 2 (PBS, pH 7.5, 1 mM MgCl₂, 2 mM DTT) the cell debris were harvested by centrifugation (3800 g, 15 min) and the protein containing supernatant was supplemented with a final concentration of 10 mM imidazole. 10 ml of Co²⁺-beads slurry (HisPur Cobalt Resin, Thermo Fisher Scientific) were washed with wash buffer (PBS, pH 7.5, 30 mM imidazole, 2 mM DTT) and the supernatant was added to the beads. After an incubation of 1 h at 4° C. the beads were washed with 20 ml wash buffer and bound protein was eluted with 20 ml elution buffer (PBS, pH 7.5, 300 mM imidazole, 2 mM DTT). Prior size exclusion chromatography (SEC), the protein elution was dialyzed overnight into PBS, pH 7.5, 2 mM DTT and concentrated via spin filter centrifugation (Amicon Ultra 15, 3 kDa, Merck Millipore).

### Flow Cytometry

Binding validation of selected phages, recombinant nanobodies was performed on Rh4-FR4 wt and Rh4-FR4ko cells. Specificity of selected phage clones binding to FGFR4 was determined by flow cytometry in 96-well plates (Becton Dickinson). Cell surface staining of Rh4-FR4 wt or Rh4-FR4ko cells was performed on ice in PBS supplemented with 1% FBS. 80 μL phages+20 μL PBS/1% milk were incubated on 1×10⁵ cells for 1 h on ice. After 2 washes in PBS, phage binding was detected by a 1:250 dilution of anti-M13 antibody (27-9420-01; GE healthcare) for 1 h on ice followed by a 1:400 dilution of A488-conjugated anti-Mouse antibody (715-545-151; Jackson ImmunoResearch, Europe Ltd) for 45 min. Samples were analyzed after two washes by flow cytometry on a MACSQuant cytometer (Miltenyi) and results were analyzed with FlowJo software (BD Biosciences, France). Phages displaying anti-mCherry nanobodies were used as negative control²⁴ and as positive control we used an anti-FGFR4 antibody (BT53, kindly provided by J. Khan lab, NCI, Bethesda, Md.). For binding test of recombinant nanobodies, cells were detached with Accutase (Stemcell Technologies) and washed with PBS. All following steps were performed on ice: 4×10⁵ cells were incubated with nanobody concentrations of 30 μg/ml for 1 h, washed once with PBS and incubated for an additional 30 min with anti His-tag FITC labeled antibody (LS-057341, LSBioscience, diluted 1:10). The cells were washed once more with PBS and analyzed. The cells were washed twice with PBS and detached with Accutase. All flow cytometry measurements were performed with Fortessa flow cytometer (BD Biosciences) and the data were analyzed using FlowJo™ 10.4.1 software.

### Western Blotting

SDS-PAGE samples were separated on 4-12% NuPAGE Bis-Tris gels (Thermo Fisher Scientific) and blotted on Trans-Blot Turbo Transfer Blot membranes (Biorad). After blocking the membranes with blocking buffer (5% milk/TBST) for 1 h at room temperature, the primary antibody was added at a 1:1000 dilution and incubated overnight at 4°. The secondary HRP-conjugated antibody was diluted 1:10′000 in blocking buffer and added to the washed membrane for 1 h at room temperature. Chemiluminescence was detected after incubation with Amersham™ ECL™ detection reagent (GE Healthcare) or SuperSignal™ West Femto Maximum Sensitivity Substrate (ThermoFisher Scientific) in a ChemiDoc™ Touch Imaging system (BioRad).

### Surface Plasmon Resonance Spectroscopy

Single cycle kinetics analysis was performed with the BIAcore T200 instrument (GE Healthcare) on CMD200M sensor chips (XanTec bioanalytics GmbH) activated with a mixture of 300 mM NHS (N-hydroxysuccinimide) and 50 mM EDC (N-ethyl-N′-(dimethylaminopropyl) carbodiimide). Recombinant FGFR1, FGFR2, FGFR3 and FGFR4 (G&P Biosciences) were immobilized on the activated biosensors (800 to 12′000 RU; 1 RU=1 pg/mm²) followed by a blocking step with 1M ethanolamine. One flow channel per chip was used as a reference to provide background corrections. The nanobodies were injected at 5 different concentrations followed by a dissociation phase. Koff-rates were determined from a final dissociation step after the last injection. The measurements with FGFR4 were performed for each nanobody on freshly immobilized protein due to strong binding and incomplete dissociation from the surface Immobilization flow rate was 5 μl/min and binding studies were performed at 30 μl/min. Binding parameters were determined with the heterogeneous ligand model fit of the BIAevaluation software. The black curves represent the measured data and red curves show the performed fit analysis.

### Sequences of Interest

| | | |
|---|---|---|
| SEQ ID | FRW1 | EVQLVESGGGLVQPGGSLRLSCAASG |
| NO: 1 | | |
| SEQ ID | FRW2 | MGWVRQAPGKGLEWVSAIS |
| NO: 2 | | |
| SEQ ID | FRW3 | YYADSVKGRFTISRDNSKNTVYLQMNSLRAEDTAVYYCA |
| NO: 3 | | |
| SEQ ID | FRW4 | YRGQGTLVTVSS |
| NO: 4 | | |
| SEQ ID | FRW1 (NA) | gaagtgcagctggtggagtccgggggaggactggtgcagccgggggggtcattgcgac |
| NO: 5 | | tgagctgcgccgcatccggg |
| SEQ ID | FRW2 (NA) | atgggctgggttcgtcaggcccctggcaaggggctggagtgggtttccgccatctcc |
| NO: 6 | | |
| SEQ ID | FRW3 (NA) | tattacgctgacagcgtaaagggaagatttacaattagccgggataactccaaaaacacgg |
| NO: 7 | | tctatctccagatgaacagcctcagggccgaggacactgcagtgtattactgtgca |
| SEQ ID | FRW4 (NA) | tatcgtggacaggggacgctggtaactgtgagtagc |
| NO: 8 | | |
| SEQ ID | Anti-FGFR4 | EVQLVESGGGLVQPGGSLRLSCAASGTGYALDDMGWVRQAPGKGLEWVSA |
| NO: 9 | Gimli_F8 | ISDDESMADYYADSVKGRFTISRDNSKNTVYLQMNSLRAEDTAVYYCASYKEY |
| | | KYQSGHHYFAYRGQGTLVTVSS |
| SEQ ID | anti_GFP_ | EVQLVESGGGLVQPGGSLRLSCAASGRFYGWYVMGWV |
| NO: 10 | Gimli_D8 | RQAPGKGLEWVSAISDQPGTEYYYADSVKGRFTISRDNS |
| | | KNTVYLQMNSLRAEDTAVYYCAHQKMHYERMYRGQGT |
| | | LVTVSS |
| SEQ ID | anti_tubulin_ | EVQLVESGGGLVQPGGSLRLSCAASGFTSERYIMGWVRQ |
| NO: 11 | Gimli_B1 | APGKGLEWVSAISRRSNYKPYYADSVKGRFTISRDNSKN |
| | | TVYLQMNSLRAEDTAVYYCALQHRYTQEMDQQHREYR |
| | | GQGTLVTVSS |

1. A method of making a synthetic single domain antibody library, said method comprising:
i. introducing a diversity of nucleic acids encoding CDR1, CDR2, and CDR3, between the respective framework coding regions of a synthetic single domain antibody to generate nucleic acids encoding a diversity of synthetic single domain antibodies with the same synthetic single domain antibody scaffold amino acid sequence,
wherein said synthetic single domain antibody scaffold comprises the following amino acid residues: FRW2-V4, FRW2-G11, FRW2-L12, FRW2-W14, FRW1-V5, FRW1-E6, FRW1-L11, FRW3-V35, FRW4-R2, FRW4-L7.

2. The method according to claim 1, wherein said synthetic single domain antibody scaffold further comprises at least one of the following amino acid residues: FRW1-P14, FRW3-S17, FRW3-R29, FRW3-A30, FRW2-S16, FRW3-K18, FRW3-V21, FRW3-Y22, FRW3-L23, FRW3-S27.

3. The method according to claim 1, wherein said synthetic single domain antibody scaffold comprises the following framework regions consisting of FRW1 of SEQ ID NO: I, FRW2 of SEQ ID NO:2, FRW3 of SEQ ID NO: 3 and FRW4 of SEQ ID NO:4, or functional variant framework regions with no more than 1, 2 or 3 conservative amino acid substitutions within each framework region with the proviso that said synthetic single domain antibody scaffold contains at least one of the amino acid residues consisting of FRW2-V5, FRW3-V21 and FRW4-R2.

4. The method according to claim 1, wherein the amino acid residues of the synthetic CDR1 and CDR2 are determined by the following rules:
at CDR1 position 1: Y, R, S, T, F, G, A, or D;
at CDR1 position 2: Y, S, F, G or T;
at CDR1 position 3: Y, S, F, or W;
at CDR1 position 4: Y, R, S, T, F, G, A, W, D, E, K or N;
at CDR1 position 5: S, T, F, G, A, W, D, E, N, I, H, R, Q, or L;
at CDR1 position 6: S, T, Y, D, or E;
at CDR1 position 7: S, T, G, A, D, E, N, I, or V;
at CDR2 position 1: R, S, F, G, A, W, D, E, or Y;
at CDR2 position 2: S, T, F, G, A, W, D, E, N, H, R, Q, L or Y;
at CDR2 position 3: S, T, F, G, A, W, D, E, N, H, Q, P;
at CDR2 position 4: G, S, T, N, or D;
at CDR2 position 5: S, T, F, G, A, Y, D, E, N, I, H, R, Q, L, P, V, W, K or M;
at CDR2 position 6: S, T, F, G, A, Y, D, E, N, I, H, R, Q, L, P, V, W, or K;
at CDR2 position 7: S, T, F, G, A, Y, D, E, N, I, H, R, Q, L, P, or V;
and wherein CDR3 amino acid sequence comprises between 9 and 18 amino acids randomly selected among one or more of the following amino acids: S, T, F, G, A, Y, D, E, N, I, H, R, Q, L, P, V, W, K, M.

5. A synthetic single domain antibody library obtainable by the method of claim 1.

6. The synthetic single domain antibody library of claim 5, comprising at least 1·10⁹ distinct antibody coding sequences.

7-8. (canceled)

9. An antigen-binding protein, comprising a synthetic single domain antibody of the following formula: FRW1-CDR1-FRW2-CDR2-FRW3-CDR3-FRW4, the framework regions consisting of FRW1 of SEQ ID NO: I, FRW2 of SEQ ID NO:2, FRW3 of SEQ ID NO: 3 and FRW4 of SEQ ID NO:4, or functional variant framework regions with no more than 1, 2 or 3 conservative amino acid substitutions within each framework region with the proviso that said synthetic single domain antibody scaffold contains at least one of the amino acid residues consisting of FRW2-V5, FRW3-V21 and FRW4-R2; optionally wherein the framework regions are derived from VHH framework regions FRW1, FRW2, FRW3, and FRW4 of Lama species.

10. The antigen-binding protein of claim 9, wherein said synthetic single domain antibody has one or more of the following functional properties:
i. it can be expressed as soluble single domain antibody in E. coli periplasm,
ii. it can be expressed as soluble intrabodies in E. coli cytosol,
iii. it does not aggregate when expressed in mammalian cell lines as fluorescent protein fusions.

11. The antigen-binding protein of claim 9, wherein the amino acid residues of the synthetic CDR1 and CDR2 are:
at CDR1 position 1: Y, R, S, T, F, G, A, or D;
at CDR1 position 2: Y, S, F, G, or T;
at CDR1 position 3: Y, S, F, or W;
at CDR1 position 5: S, T, F, G, A, W, D, E, N, I, H, R, Q, or L;
at CDR1 position 6: S, T, Y, D, or E;
at CDR1 position 7: S, T, G, A, D, E, N, I, or V;
at CDR2 position 1: R, S, F, G, A, W, D, E, or Y;
at CDR2 position 2: S, T, F, G, A, W, D, E, N, H, R, Q, L or Y;
at CDR2 position 3: S, T, F, G, A, W, D, E, N, H, Q, P;
at CDR2 position 4: G, S, T, N, or D;
at CDR2 position 5: S, T, F, G, A, Y, D, E, N, I, H, R, Q, L, P, V, W, K or M;
at CDR2 position 6: S, T, F, G, A, Y, D, E, N, I, H, R, Q, L, P, V, W, or K;
at CDR2 position 7: S, T, F, G, A, Y, D, E, N, I, H, R, Q, L, P, or V;
and wherein CDR3 amino acid sequence comprises between 9 and 18 amino acids selected among one or more of the following amino acids: S, T, F, G, A, Y, D, E, N, I, H, R, Q, L, P, V, W, K, M.

12. The antigen-binding protein of claim 9, which further comprises an F-box domain for targeting a protein to the proteasome.

13. An isolated nucleic acid that encodes an antigen-binding protein of claim 9.

14. The isolated nucleic acid of claim 13 comprising the following nucleic acid sequences SED ID NO:5, SEQ ID NO:6, SEQ ID NO:7, SEQ ID NO:8 encoding respectively framework regions FRW1, FRW2, FRW3 and FRW4 of SEQ ID NO:1-4.

15. A method of producing the antigen binding protein of claim 6 in a recombinant host cell comprising:
i. culturing a host cell comprising a nucleic acid encoding the antigen-binding protein under appropriate conditions for the production of the antigen-binding protein, and
ii. isolating said antigen binding protein.
